(19) United States
(12) Patent Application Publication
(10) Pub. No.: US 2005/0277910 A1
(43) Pub. Date: Dec. 15, 2005

(54) Catheter tube

(75) Inventors: Andreas Dolla, Hof (DE); Udo Gobel, Melsungen-Kirchhof (DE)

Correspondence Address: VENABLE LLP, P.O. BOX 34385, WASHINGTON, DC, 20045-9998, US

(73) Assignees: Rehau AG & Co., Rehau (DE); B. Braun Melsungen AG, Melsungen (DE); Raumedic AG., Munchberg (DE)

(21) Appl. No.: 10/513,673

(22) PCT Filed: Apr. 30, 2003
(86) PCT No.: PCT/EP03/04516

§ 371 Date: Nov. 8, 2004

(30) Foreign Application Priority Data
May 8, 2002 (DE) 102 20 409.8

(51) Int. Cl. A61M 25/098

(52) U.S. Cl. 604/529; 137/141

(57) ABSTRACT

The invention relates to a catheter tube for local anaesthetic with improved characteristics in terms of resistance to kinking, a higher degree of rigidity during application and reduced rigidity after application. According to the invention, said characteristics are achieved by designing the catheter tube as a layered composite tube, whereby the interior that faces the lumen consists of a polyamide layer and the exterior consists of a polyurethane layer. The layer thickness distribution of the polyamide inner layer to the polyurethane outer layer is preferably between 50:50 and 70:30. The inner and outer layers of the inventive catheter tube can be provided with one or more radiopaque strips.

Catheter tubes which are mono-tubes with or without radiopaque strips for use in the field of regional anesthesia are prior art.

For this purpose materials, such as PA, PUR, PTFE, PE, PVC and silicone are utilized which, based on their properties as related to regional anesthesia, are to be classified as follows:

| | chemical resistance | flexural | kinking | tear | expan- | trans- |
|---|---|---|---|---|---|---|
| material | to anesthetics | strength | resistance | resistance | sion | parency |
| PVC | − | 0 | 0 | 0 | 0 | + |
| PE | + | 0 | − | + | + | − |
| silicone | + | − | + | 0 | + | − |
| PA | + | + | 0/+ | + | 0 | + |
| PTFE | + | − | − | + | 0 | − |
| PUR | + | 0 | + | + | + | + |
| FEP | + | − | − | + | + | − |

Additional information as regards the indication, application and medical requirements may be gleaned from the following bibliography:
Niesel, Hans Christoph Regionalanåsthesie Lokalanasthesie Regionale Schmerztherapie(Regional Anesthesia, Local Anesthesia, Pain Therapy); George Thieme Publisher; Stuttgart, N.Y.; 1994, 291-350;
Hahn, B. H. Regionalanåsthesie (Regional Anesthesia); NcQuillan, M. Ullstein Medical Verlagsgesellschaft mbH & Sheplock, G. J. Co., Wiesbaden, 1999, 209-215
Jankovic, Danilo Regional Nerve Blocks; Wells, Christopher Blackwell Science; Berlin, Vienna, 2001, 243-252
Moor, D. C. Regional Blocks; Charles C. Thomas; Springfield, Ill., USA, Fourth Edition, 1979, 427-438
Saint-Maurice Regional Anesthesia in Schulte Steinberg Children; Appleton & Lange/Mediglobe; Norwalk/San Mateo/Fribourg, 1990, 106-109
Scott, D. B. Handbook of Epidural Anaesthesia and Analgesia; Schulz Medical Information; Copenhagen, Denmark, 1985, 86-99.

In the examples which follow, flexural strength, feeding ease, resetting force and kinking resistance, that is, properties, which are of significance for the regional anesthesia, will be discussed with a view of the manner in which the properties of the materials may affect the application of the catheter tube in the patient.

Flexural Strength

If the catheter tube is too soft, it may not be well positioned in the epidural space, particularly in case of difficult anatomic conditions. Further, a risk of faulty positioning exists, particularly a lateral aberration that may culminate in a loop or knot formation. An excessively hard catheter tube involves a high risk of intravasal or intrathecal positioning as well as nerve irritations which may be reversible or irreversible.

Resistance to Kinking

A low kinking resistance involves the danger of a blockage of the catheter tube with the consequence that the patient is only insufficiently supplied with analgesics, if at all, or a pumping alarm may be triggered in case infusion pumps are used for the application of anesthetics.

Ease of Feed

If the catheter tube has an insufficient ease of feed, it may be placed in the epidural space through the epidural canulae in an unsatisfactory manner, or cannot be placed at all. This leads to prolongations of the application time and an unnecessary burden on the patient or to a selection of another anesthesia technique.

Resetting Force

An insufficient resetting force of the catheter tube leads to faulty positions because of a low or missing tendency of the catheter tube to assume its original position prior to its deflection.

In the present prior art it is of particular disadvantage that the mechanical properties of the raw materials significantly fluctuate because of manufacturing conditions. This manifests itself primarily in the Shore hardness data provided by the suppliers of raw materials. Tolerances of ±4 units are usual. For example, in case of PUR, a given Shore hardness of 60 D may in practice lie between 56 D and 64 D which has a significant effect on the mechanical properties of the catheter tube, such as flexural strength, involving the above-outlined negative considerations. The mechanical properties of the catheter mono-tube thus cannot be set in a reproducible or in a specific (or quasi-stepless) manner.

It is a further disadvantage of the prior art that the medically highly desirable property of a catheter, namely that upon application, that is, upon introduction into the body, it is rigid to the extent possible, but during the period it dwells in the body, however, it is flexible to the extent possible in order to prevent inflammations, can be achieved with mono-tubes only in a very limited manner.

It is accordingly the object of the invention to provide a catheter tube having the following properties:
a constant or specific property level, particularly as concerns the mechanical properties,
a high degree of rigidity during feeding the catheter tube, eventually resulting in a satisfactory ease of feed,
a flexible behavior for minimizing intravasal and intrathecal positions as well as nerve irritations,
a high degree of resistance to kinking,
a reduction of the rigidity after positioning the catheter tube for minimizing hematoma formations,
an absorption, to the smallest extent possible, with respect to the usual local anesthetics, and
preserving the mechanical and biological properties in extended use.

In the description which follows, considerations which lead to achieving the object of the invention will be set forth:

For making tubular extrusions, thus also for medical purposes, homo-polymers, but mostly co-polymers or polymer mixtures are used. If, however, these commercially available standard materials are insufficient as concerns their property spectrum, a more significant, recipe-conditioned outlay is required for obtaining the desired property modification.

In cases where the inner surface of the tube has to exhibit properties which are significantly different from those of the outer surface—for example, in case the outer layer is required to have good adhesion-capable properties and the inner surface should exhibit a good resistance to local anesthetics—a recipe-based manufacture is no longer expedient. In such a case one readily turns to layer composites having materials of different properties. The macroscopic properties of such layer composites, such as rigidity or flexural strength are, as a rule, determined by the sum of the participating material properties.

For example, U.S. Pat. No. 4,385,632 describes a layer composite for an angiographic catheter. The catheter tube has a soft, non-traumatic tip made exclusively from polyurethane, a transitional region and a main tube made from a polyamide inner layer and a polyurethane outer layer at an approximate wall thickness ratio of 50:50. The purpose of such a structure as a layer composite is to fulfill the following requirements:
to make possible a soft, non-traumatic, non-reinforced polyurethane tip,
to anchor it firmly to the catheter tube,
to ensure a correspondingly high strength of the catheter at a small wall thickness. Such high strength is required, since the X-ray contrast substance is applied at a high pressure (up to 1200 psi) and, at the same time, a bursting of the catheter must be securely prevented.

In contrast, the present invention seeks to achieve catheter properties in the field of regional anesthesia as stated earlier in accordance with the formulation of the object of the invention. This is only possible if beyond the substance properties of the individual layers new property combinations are obyained which significantly exceed the sum of the individual properties. This may be achieved only if the systems exhibit a certain synergistic effect, and furthermore, the used materials are accurately coordinated with the given instance of application.

The object was unexpectedly achieved by forming the catheter tube by a layer composite of a PUR outer layer and a PA inner layer. The polyamide is selected from the group of polyamide 11, polyamide 12 or polyether block amide and the polyurethane is selected from the group of polyether polyurethane or polyester polyurethane.

In the description which follows the properties of the invention will be set forth in greater detail.

As noted earlier, in the medical field it is a very distinct desire that the catheter tube be rigid to the possible extent during introduction into the body, but be flexible while it dwells in the body. The properties which appear to be mutually exclusive, may be unexpectedly achieved by the material combination according to the invention: PUR becomes flexible and soft primarily by the effect of temperature. This event is completed within a few minutes, already at body temperature. For example, an initial Shore hardness sinks at 37° C. from 60 D to 50 D within about 5 minutes. In contrast, the flexibility of the PA is affected exclusively as a result of moisture intake; this occurrence lasts substantially longer as compared to the PUR.

It is particularly advantageous that as a function of the relative wall thickness of PUR to PA and the utilized Shore hardness, on the one hand, the rigidity for inserting the catheter tube and, on the other hand, the flexibility needed for its dwelling in the body may be accurately set. Further, by virtue of the relative layer thicknesses of the two polymers and the selection of standard types with different Shore hardness not only the flexibility/softness, but also further properties important for the application of the invention may be specifically set, namely flexion strength, resistance to kinking, ease of shifting and resetting behavior. Such a possibility is not given in the presently used mono-tube anesthesia catheters according to the prior art.

According to the invention, even in case of fluctuations of the raw material quality, the desired properties of the catheter tube may be advantageously obtained to a large extent by varying the layer thicknesses of the co-extruded tube. Further, by the utilization of current standard materials having different mechanical properties, it is feasible to significantly change—yet in a simple manner—the overall properties of the catheter tube without needing special, recipe-based mixtures.

Such recipe-based mixtures, however, are additionally feasible according to the invention and may effect a further optimization of properties, or a fine tuning of the catheter properties.

In the application for regional anesthesia three ranges of flexural strength have been found to be optimal for the different fields of use:

Range of Flexural Strength

I. 80-90 mN

II. 170-180 mN

III. 300-335 mN

The total wall thickness for medical requirements may be basically determined by the parameters
wall thickness of the inner tube,
wall thickness of the outer tube,
Shore hardness of the PA inner tube and
Shore thickness of the PUR outer tube in such a manner that in each instance the desired macroscopic range of flexural strength of the catheter tube is obtained.

For example, using for a catheter tube, having a dimension of 0.85×0.45 mm and a flexural strength II, a PA type having a Shore hardness of 74 D for the an inner tube and a PUR type having a Shore hardness of A 85, there is obtained a wall thickness of 0.08 mm for the inner tube and a wall thickness of 0.12 mm for the outer tube, that is, a wall thickness distribution of 40% PA to 60% PUR.

According to the invention, however, PA to PUR wall thickness ratios of 50:50 to 70:30 are preferred, because in this manner not only the desired flexural strength is obtained, but also an optimum for most applications, as concerns resistance to kinking, ease of feed and resetting force.

By means of the inventive composite tubes formed of a combination of a PA inner tube with a PUR outer tube it is thus possible to obtain not only the actual properties of PA mono-tubes, but also to achieve significant improvements in quality and properties as may be seen from the embodiment which follows.

The invention will be discussed in more detail by describing an anesthesia catheter without radiopaque strips.

In this example the anesthesia catheter has a dimension of 0.85×0.45 mm, that is, an outer wall thickness of 0.85 mm and an inner diameter of 0.45 mm and thus a wall thickness of 0.20 mm.

The wall thickness distribution is 30% PUR outer layer, corresponding to 0.06 mm and 70% PA inner layer, corresponding to 0.14 mm.

Considering the dependencies of the flexural strength of the overall system, that is, of the catheter tube, as a function of the Shore hardness of the respective materials, the following result is obtained:

| example | material combination | flexural strength | |
|---|---|---|---|
| 1 | PA 70D/PA 70D | 110-130 | mN |
| 2 | PA 70D/PUR 55D | 60-65 | mN |
| 3 | PA 70D/PUR 60D | 80-90 | mN |
| 4 | PA 70D/PUR 65D | 130-150 | mN |
| 5 | PA 70D/PUR 68D | 170-180 | mN |
| 6 | PA 70D/PUR 70D | 200-220 | mN |

The Table thus shows, for example, that by preserving the Shore hardness of a pure PA catheter, the flexural strength may increase by 50% if, instead of PA an outer layer of PUR is selected (compare examples 1 and 6). At the same time, the already mentioned effect appears, namely, that a short time after application, under the effect of body temperature, the catheter tube according to the invention becomes significantly softer than a pure PA catheter tube.

The Table which follows shows, for a second dimensioning of the catheter tube, in this case 1.05 mm×0.60 mm, the dependency of the flexural strength as a function of the PUR Shore hardness, wherein the ratio of the PA inner layer to the PUR outer layer remains unchanged 70% to 30%.

| example | material combination | flexural strength |
|---|---|---|
| 7 | PA 70D/PUR 60D | 250-290 nM |
| 8 | PA 70D/PUR 68D | 300-335 nM |
| 9 | PA 70D/PUR 70D | 410-430 nM |

A comparison of the examples 6 and 9 is of interest; it is seen therefrom how strongly the flexural strength of the catheter tube depends from the dimension—at a Shore hardness set at 70 D.

1. A catheter tube made of polymers for use in the field of regional anesthesia, formed of a layer composite of polyamide and polyurethane, wherein the inner side facing the lumen is formed of a polyamide layer and the outer side is formed of a polyurethane layer.

2. The catheter tube as defined in claim 1, characterized in that the polyamide is selected from the group of polyamide 11, polyamide 12 or polyether block amide.

3. The catheter tube as defined in claim 1, characterized in that the polyurethane is selected from the group of polyether polyurethane or polyester polyurethane.

4. The catheter tube as defined in claim 1, characterized in that the layer composite is a co-extrusion.

5. The catheter tube as defined in claim 1, characterized in that the layer composite has a layer thickness distribution of the PA inner layer to the PUR outer layer as 20:80 to 80:20, preferably 50:50 to 70:30.

6. The catheter tube as defined in claim 1, characterized in that the inner and/or outer layer has one or more radiopaque strips.
